(19) United States
(12) Patent Application Publication
(10) Pub. No.: US 2007/0270716 A1
(43) Pub. Date: Nov. 22, 2007

(54) Disposable Urine Collector

(76) Inventors: Chen Hung Wu, Yilan City (TW); Jia Hao Wu, Yilan City (TW)

Correspondence Address: Eric Chan, 42 Pin Oaks Drive, Phoenixville, PA, 19460, US

(21) Appl. No.: 11/381,131

(22) Filed: May 2, 2006

(51) Int. Cl. A61B 5/00

(52) U.S. Cl. 600/580

(57) ABSTRACT

A disposable urine collector which includes a slanted collecting cup with an upper opening, a urine reservoir connected to the collecting cup, and a saw-toothed flexible neck located between the collecting cup and the urine reservoir; wherein the collecting cup combined with the flexible neck which allows the collector to fit the female's genitals and assures stability when in use. In addition, a wrapping material can be wrapped around the rim of the collecting cup to provide a soft feel and to prevent the leakage of urine. The urine reservoir is filled with absorbent material. A cotton tube is extended from the top of the urine reservoir to the lower area of the urine reservoir. On one side of the collecting cup is installed is removable sticking tape. When the collector has been used, the collecting cup can be squeezed and folded to one side of the collecting cup, then one end of the sticking tape can be peeled off and stuck on the body of the urine reservoir for disposal.

### BACKGROUND OF THE INVENTION

The present invention relates to an improved urine collector, especially for use at places without a toilet, such as outdoors or in a car. The present invention provides a urine collector which is ergonomic and has the advantages of simplicity and disposability.

Due to the traffic situation, especially during rush hour or a holiday, the driver and passengers of a car usually have to suffer a long time without a restroom, and this often happens when there is a traffic jam on the highway. Furthermore, considering the physiological differences of females, suppressing the urge to urinate for a long time can easily cause infections or diseases of their urethra or bladder. Moreover, because public restrooms are often unavailable in remote areas, people are forced to suppress the urge to urinate, especially for a female who is too shy to solve her problem outdoors.

Taiwanese utility model patent number M260227 was issued to Kowoo Co., Ltd. and published on Apr. 1, 2005. It disclosed a children's urine collection device made of waterproof cardboard and comprising a long vertical board and a horizontal extended board perpendicular to each other at a 90-degree angle. The edges of the combination are folded inward to shape the device into a urine-receiving container, and the inner space constituted by said vertical and horizontal boards is spread with absorbent material to absorb the urine.

The described prior art, although it provides a urine-collecting device made of folding cardboard, still has some defects which make it uncomfortable for a woman's use. The defects include the following.

1. The prior art is designed to form a perpendicular cardboard structure in accordance with the physiological differences between males and females and with the liquid flow of urine. That is to say, when the device is used by a female, the longer vertical board can be laid down to let the female urinate in a squatting position. However, when the device is used by a woman is in a car, it is impossible to urinate in a squatting position. Meanwhile, most women could not accept urinating in squatting position. Thus, the prior art cannot solve the problem for a woman when she needs to urinate in a car.

2. The prior art disclosed folded edges on its vertical and horizontal cardboard members to prevent the splashing of the urine. However, because there exists a gap between the device and the genitals of the male or the female, there is no guarantee that the urine would not splash out and thus fail to be collected in the device.

3. Because the prior art is designed to form an open structure, when the device is used, it is easy for urine to leak out during the disposal process. Also, because the device is designed to form an open structure, it is easy for odors to spread after its use. While deodorant could be added to the absorbent material, it would still be hard to prevent the odor from spreading.

U.S. Pat. No. 4,457,314 issued on Jul. 3, 1984, claims a female urine collection device and method which includes a cup-shaped receiving chamber with a flexible lip which seals around the urethral orifice. A duct section leads to flexible tubing leading to a compressible collection reservoir. The chamber and reservoir are mounted in a housing of soft absorbent material.

The abovementioned prior art disclosed a urine collection device for females, and more particularly for incontinent females. However, it is not easy to use in a car and is uncomfortable to hold when used.

The present invention intends to provide an improved urine collector to mitigate or obviate the aforementioned problems.

### BRIEF SUMMARY OF THE INVENTION

The primary object of the present invention is to provide a urine collector which is easy to use, comfortable and disposable. The urine collector of the present invention is an organic whole comprising a slanted collecting cup with an upper opening, a urine reservoir connected to the collecting cup, and a saw-toothed flexible neck located between the collecting cup and the urine reservoir.

The urine reservoir is filled with an adequate absorbent material, such as a high-polymer absorbent core. A cotton tube is extended from the top of the urine reservoir to the lower area of the urine reservoir to lead the urine to the lower area of the urine reservoir and thereby prevent the urine from overflowing the urine reservoir due to the absorbent material absorbing the urine too slowly when a great quantity of urine suddenly flows into the reservoir.

On one side of the collecting cup there is removable sticking tape. When the collector has been used, the collecting cup can be squeezed and folded to that side of the collecting cup, then one end of the sticking tape can be peeled off and stuck on the body of the urine reservoir to secure it for disposal. Furthermore, when the urine collector has been used, the user pushes the collecting cup along its tapered edge and squeezes the collecting cup flat, whereby a triangular piece is produced, the arc bottom of which seals the opening of the flexible neck to prevent urine backflow from the urine reservoir.

In addition, the collecting cup of the present invention provides a slanted structure, and a saw-toothed flexible neck is located between the collecting cup and the urine reservoir. The aforesaid design allows the urine reservoir to be adjusted to conform to the collecting cup, which means the cylinder-like urine reservoir can be maintained in a perpendicular position by gravity, and because the urine reservoir is adjustable to conform to the collecting cup, the present invention can stay steady when it is used in a vibrating environment.

By virtue of the structures stated above, the present invention includes the following advantages:

1. The present invention provides a collecting cup that is ergonomic and can completely fit the female's genitals and collect urine effectively while preventing the urine splashing out.

2. Because the collecting cup of the present invention is placed against the female's genitals, no matter whether the present invention is used in a car or any other narrow space, it is still easy to use. Furthermore, because the present invention has a small volume and is used directly against the female's genitals, it is convenient and discreet.

3. The collecting cup of the present invention provides a slanted structure and a saw-toothed flexible neck located between a slanted collecting cup and a cylinder-like urine reservoir. When the present invention is used in a car, the slanted collecting cup can completely conform to the female's genitals, so that the cylinder-like urine reservoir can maintain a perpendicular position by gravity. The aforesaid design is not only easy for a female user to grasp by hand, but the flexible neck can absorb the dynamic energy caused by the vibrating of the car, so that the collecting cup would not come away from the female's genitals. Moreover, because the urine reservoir can be grasped by the user's hand, the present invention is more steady and safe when in use.

4. After the present invention has been used, it can be squeezed and folded, using sticking tape to secure the folding. The design is not only convenient for disposal, but also can prevent the odor of the urine spreading out into the car.

5. The present invention is designed for females in accordance with their unique physiological structure, and provides a urine collector which is ergonomic. As for males, the present invention also provides a convenient and easy-to-use design.

Furthermore, the rim of the collecting cup of the present invention defines a curving lip which not only provides a supportive contour to the collecting cup, but also provides a soft body-conforming surface to prevent any leakage of urine. On that curving lip, it is also optional to wrap a layer of wrapping material, such as non-woven fabric, to form a web layer. Meanwhile, the layer also extends inward and downward from the top of the collecting cup to the lower area of the collecting cup. Such a web layer is not only absorbent and comfortable in use, but when after use of the urine collector the user pushes the collecting cup along its tapered edge and squeezes the collecting cup flat, whereby a triangular piece is produced, the arc bottom of which can seal the opening of the flexible neck as stated above, to prevent urine backflow from the urine reservoir, and the web layer which covers the arc bottom of the triangular piece can also seal the opening of the flexible neck and wipe away any residual urine. Therefore, the present invention provides three levels of protection to prevent any possibility of leakage of urine: a high-polymer absorbent core in the urine reservoir, an arc bottom of the triangular piece, and the sealing of the opening of the flexible neck by using the wrapping material when the present invention has been squeezed and folded to resize the collector.

Other objects, advantages and novel features of the invention will become more apparent from the following detailed description when read in conjunction with the accompanying drawings.

### BRIEF DESCRIPTION OF THE DRAWINGS

FIG. 1 is a perspective view of the present invention;

FIG. 2 is a perspective view of the present invention illustrating the act of squeezing the collecting cup;

FIG. 3 is a perspective view of the present invention illustrating the act of folding the collecting cup;

FIG. 4 is a side view of the present invention;

FIG. 5 is a plan view of the present invention illustrating the position of the collecting cup in use;

FIG. 6 is a side view of the present invention illustrating the position of the triangular piece;

FIG. 7 is a perspective view of the present invention illustrating the web layer on the rim of the collecting cup;

FIG. 8 is a vertical plan view of the present invention illustrating the covering area of the web layer;

FIG. 9 is a side view of the present invention illustrating the web layer on the rim of the collecting cup; and

FIG. 10 is a side view of the present invention illustrating the position of the triangular piece with a web layer.

### DETAILED DESCRIPTION OF THE INVENTION

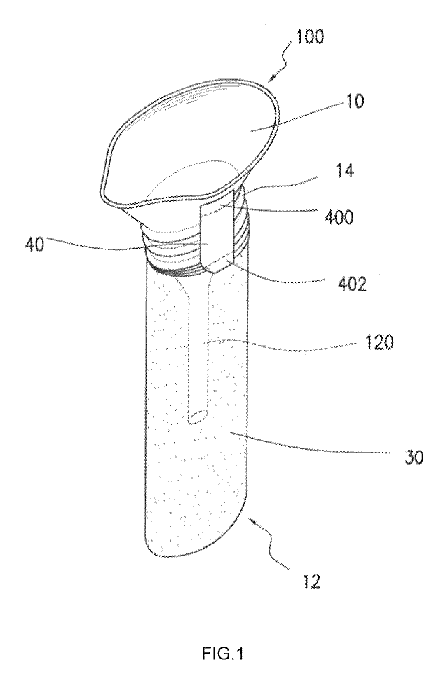

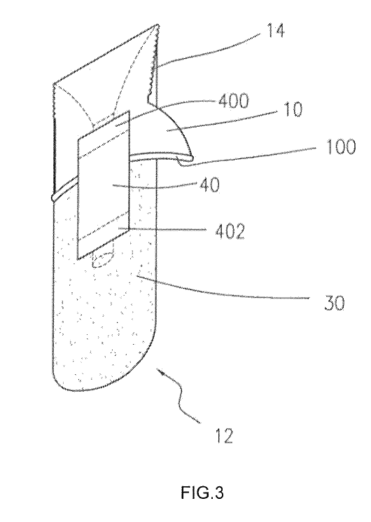

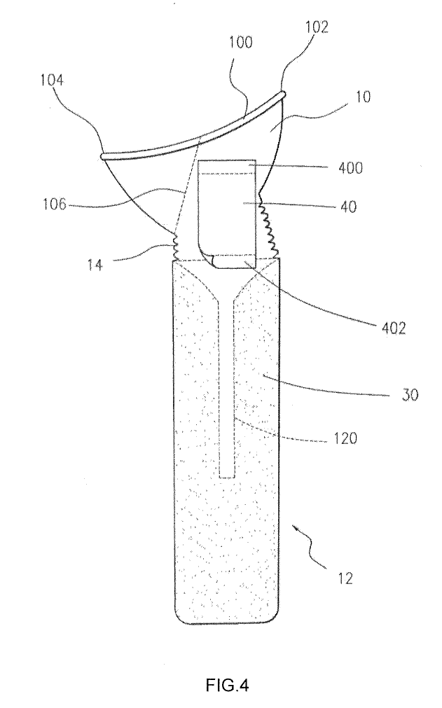

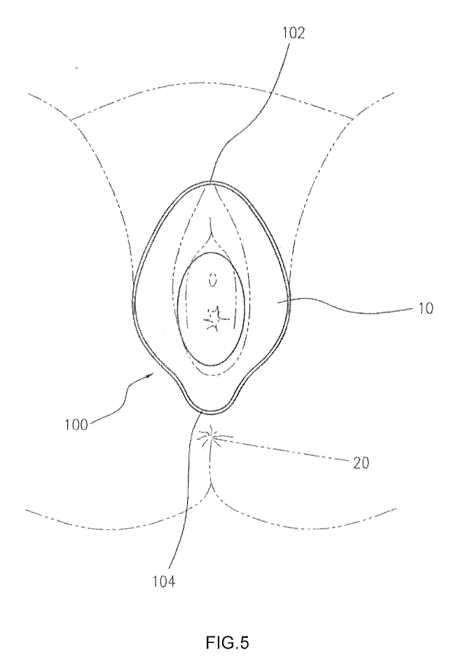

As shown in FIGS. 1 to 5, the present invention is a design encompassing an organic whole and having features comprising a slanted collecting cup 10 with an upper opening, a urine reservoir 12 connected to the collecting cup 10, and a saw-toothed flexible neck 14 located between the collecting cup 10 and the urine reservoir 12. Wherein, the top opening of the collecting cup 10 is shaped like a watermelon seed, referring to FIG. 5, the front side of the opening of the collecting cup 10 is formed into an oval-shaped edge 102, and the rear side of the opening of the collecting cup 10 is formed into a tapered edge 104. The rim 100 of the opening is designed to form a curving lip which not only provides a supportive contour to the collecting cup 10, but also provides a soft body-conforming surface to prevent the leakage of urine. In addition, when the aforesaid collecting cup 10 in the shape of a watermelon seed is used, the oval-shaped edge 102 is in the front, and the tapered edge 104 is in the rear, and referring to FIG. 5, the tapered edge 104 in the rear is located in front of the anus 20, so as to prevent any infection by bacteria. As to the collecting cup 10, it has been ergonomically designed to form a slanted angle to apply to the genital position of females.

As shown in FIG. 1, the urine reservoir 12 is a cylinder-like design made of waterproof and leakage-proof materials, e.g., PE (polyethylene). The reservoir is filled with an adequate high-polymer absorbent core 30, and a funnel-like cotton tube 120 is defined in the urine reservoir 12, with its top extended to the lower area of the urine reservoir 12. The cotton tube 120 is made of cotton paper pulp and has the nature of capillarity, and can lead the urine to the absorbent core 30 in the lower area, thereby ensuring that the urine has been effectively absorbed by the absorbent core 30 and preventing the urine from concentrating in the upper area of the urine reservoir 12 when a great quantity of urine has flowed into the urine reservoir 12 and the absorbent core 30 in the upper area absorbs the urine too slowly, causing the urine to overflow from the collecting cup 10.

The saw-toothed flexible neck 14 located between the collecting cup 10 and the cylinder-like urine reservoir 12 is flexible and retractable so that the urine reservoir 12 can be flexibly adjusted in accordance with collecting cup 10, which means that the urine reservoir 12 can be maintained in the perpendicular by gravity when the collector is used. In the light of the foregoing, the urine collector can be used in a vibrating environment, and the stability of the urine collector can be assured. For example, when the present invention is used in a car, the slanted collecting cup 10 can be completely placed against the female's genitals, while the cylinder-like urine reservoir 12 can be maintained in the perpendicular by gravity, to allow the user to grasp it. Due to the design of the flexible neck 14, the urine reservoir 12 can be maintained in the perpendicular by gravity, the collecting cup 10 can be placed against the female's genitals, and the vibrating dynamic energy can be absorbed by virtue of the saw-toothed flexible neck 14. The aforesaid design makes it not only easy for a female user to grasp the urine reservoir 12 by hand in a perpendicular position, but also assures that the collecting cup 10 would not come away from the genitals, so that stability and safety are assured.

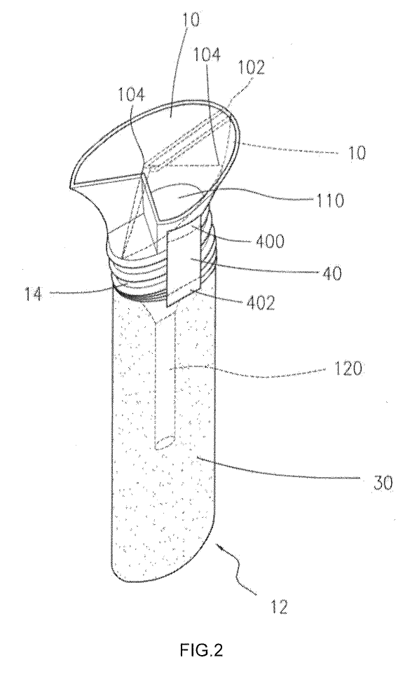

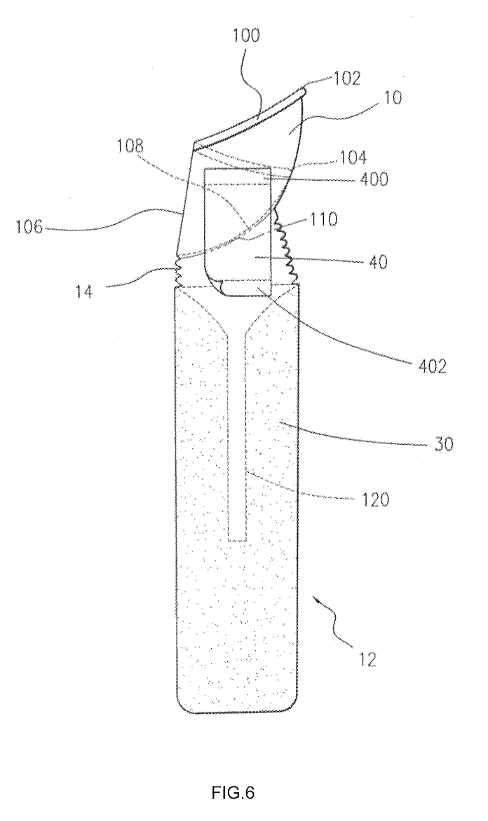

Furthermore, with reference to FIGS. 1 to 4, on one side of the collecting cup 10 is installed removable sticking tape 40, the upper end of which is a fixed end 400 and the lower end of which is a removable end 402. The removable end 402 can be peeled off in opposition to the fixed end 400. After the collector has been used, the collecting cup 10 can be squeezed and folded for disposal and to prevent the odor from spreading. As shown in FIGS. 2 to 4, when the collector has been used, because of a tapered edge 104 is defined on one side of the collecting cup 10, the user can push the tapered edge 104 forward in the direction of the oval-shaped edge 102, and then the tapered edge 104 can be folded to become an inner triangular piece. Deserving of mention is that, for convenience in forming a triangular piece, the folding lines 106 can be pre-pressed along the folding position of the tapered edge 104 to make the folding of the tapered edge 104 much easier and faster. When the tapered edge 104 has been pushed forward to become an inner triangular piece, the opening of the collecting cup 10 can be squeezed to be flat, and the opening of the collecting cup 10 to form a sealed alignment ( as dotted lines shown in FIG. 2). When the tapered edge 104 has been pushed forward to become an inner triangular piece, an arc bottom 108 defined on the bottom of the triangular piece can seal the opening 110 of the slanted flexible neck 14, which is installed above the urine reservoir 12 (as shown in FIG. 6), to prevent the urine backflow from the urine reservoir 12. In view of foregoing, when the opening of the collecting cup 10 has been squeezed to be flat, and folded the collecting cup 10 to its one side, then the lower removable end 402 of the sticking tape 40 can be peeled off and removed in opposition to the fixed end 400, to stick on the body of the urine reservoir 12 at the time of the collecting cup 10 has been folded, to secure the folding. The design, it is not only to reach the purpose to seal with the opening of collector, but also to reduce the size of the collector in order for disposal.

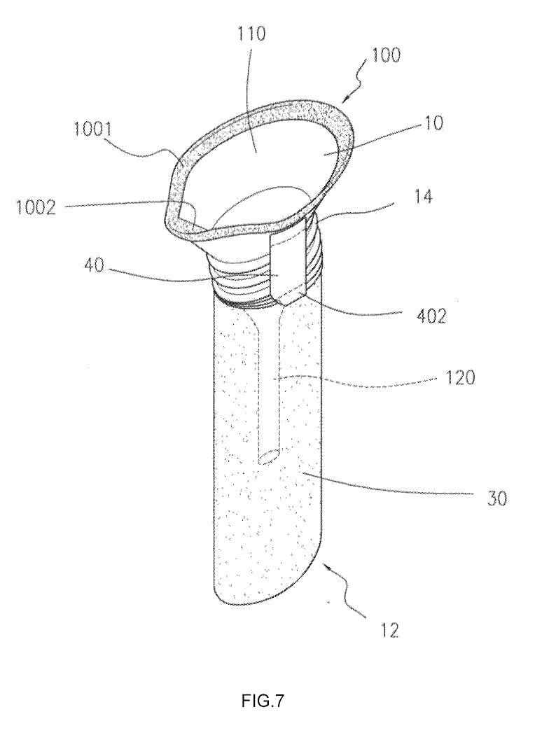

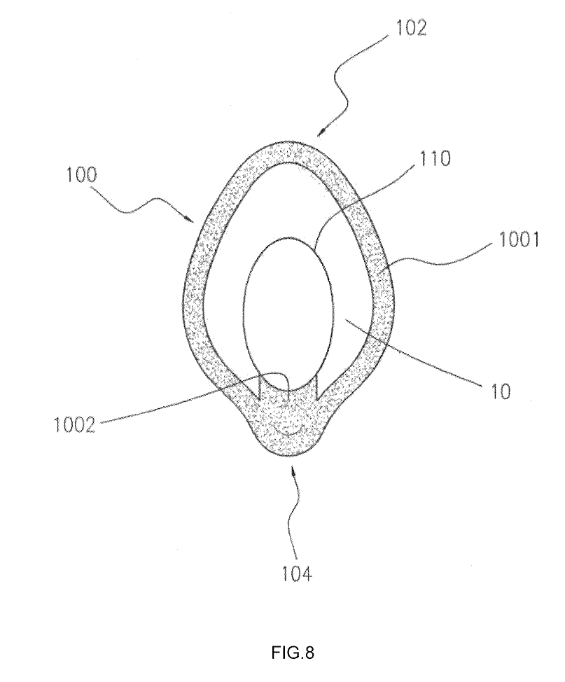

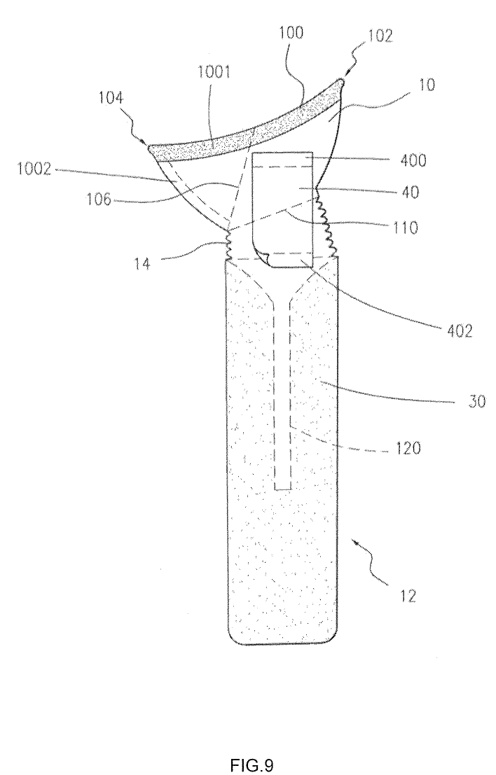

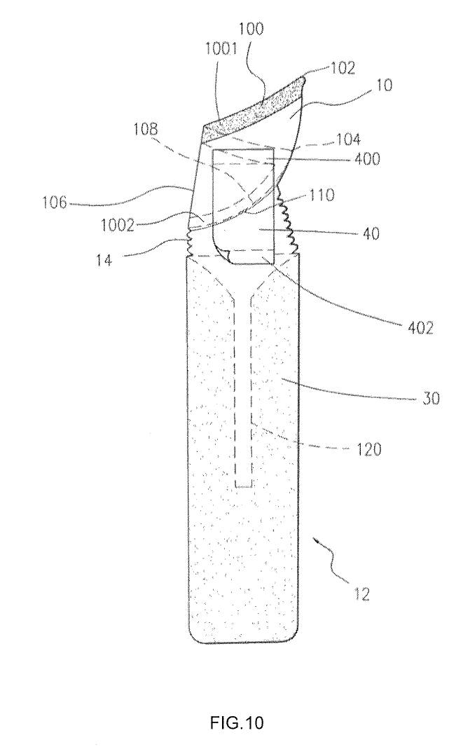

As for the rim 100 of the collecting cup 10 of the present invention, it is optional to wrap a layer of wrapping material, such as non-woven fabric on the curving lip of the rim 100, to form a web layer 1001. Referring to FIGS. 7 to 10, the wrapping material also extends to the inward and downward area of the collecting cup 10, from the tapered edge 104 of the collecting cup 10 down to the lower opening 110 of the collecting cup 10, to form a covering layer 1002 (As shown in FIGS. 8 and 9). When a female uses the collecting cup 10 of the present invention to attach to her genitals, the web layer 1001 on the rim 100 provides a comfortable and soft touch to the skin around the genitals, meanwhile when the collecting cup 10 has been used, the user pushes the collecting cup 10 from its tapered edge 104 and squeezes the collecting cup 10 to be flat, a triangular piece that produced by pushing the tapered edge 104, the arc bottom 108 of the triangular piece which with a covering layer 1002 therefore can seal the lower opening 110 of collecting cup 10, which is installed above the slanted flexible neck 14 (as shown in FIG. 10). Therefore, the present invention has provided a structure to prevent the urine backflow from lower opening 110 of the collecting cup 10 by using the arc bottom 108 of the triangular piece as described above, optionally the covering layer 1002 which covers on the arc bottom 108 of the triangular piece can also seal the lower opening 110 of the collecting cup 10, and further absorb and wipe away the residual urine from any leakage. In view of foregoing, the present invention provides three levels of protection to prevent any possibility of leakage of urine: the high-polymer absorbent core 30 in the urine reservoir 12, the arc bottom 108 of the triangular piece, and the sealing of the opening 110 of the flexible neck 4 by using the wrapping material when the present invention has been squeezed and folded to resize the collector.

Although the present invention is designed in accordance with the female's unique physiological structure, it is not limited to female use only. Male users also can use the present invention without any obstacle since they have a more convenient physiological structure than females.

Even though numerous characteristics and advantages of the present invention have been set forth in the foregoing description, together with details of the structure and function of the invention, the disclosure is illustrative only, and changes may be made in the details, especially in matters of shape, size, and arrangement of parts within the principles of the invention to the full extent indicated by the broad general meaning of the terms in which the appended claims are expressed.

What is claimed is:

1. A disposable urine collector comprising:
a slanted collecting cup with an upper opening;
a cylinder-like urine reservoir connected to the collecting cup; and
a saw-toothed flexible neck located between the collecting cup and the urine reservoir;
wherein the top opening of the collecting cup is shaped like a watermelon seed, with one side of the opening of the collecting cup formed as an oval-shaped edge, and the other side of the opening of the collecting cup formed as a tapered edge; in
addition, the collecting cup is designed to form a slanted angle which conforms to the genital position of females, and the urine reservoir is maintained in the perpendicular by gravity.

2. The urine collector as claimed in claim 1, wherein the collecting cup, the urine reservoir, and the flexible neck comprise a design encompassing an organic whole.

3. The urine collector as claimed in claim 1, wherein the urine reservoir is a cylinder-like design made of waterproof and leakage-proof materials.

4. The urine collector as claimed in claim 3, wherein the urine reservoir is filled with adequate absorbent material to absorb the urine which is led to the urine reservoir.

5. The urine collector as claimed in claim 1, wherein a funnel-like cotton tube is installed in the urine reservoir and extends from the top to the lower area of the urine reservoir.

6. The urine collector as claimed in claim 5, wherein the cotton tube is made of cotton paper pulp and has the nature of capillarity.

7. The urine collector as claimed in claim 1, wherein on one side of the collecting cup is installed removable sticking tape, the upper end of which is a fixed end, and the lower end of which is a removable end, and the removable end can be peeled off in opposition to the fixed end and stuck on one side of the urine reservoir.

8. The urine collector as claimed in claim 1, wherein the collecting cup has pre-pressed folding lines along the folding position of the tapered edge; when the tapered edge has been pushed forward to become an inner triangular piece, the opening of the collecting cup can be squeezed flat, the opening of the collecting cup then forms a sealed alignment; an arc bottom is defined on the bottom of the triangular piece, and the arc bottom can seal the opening; when the opening of the collecting cup has been squeezed flat and folded to one side of the collecting cup, the folding can be secured by using sticking tape.

9. The urine collector as claimed in claim 1, wherein said collecting cup has the shape of a watermelon seed; when it is used, the oval-shaped edge is in the front, and the tapered edge is in the rear, the tapered edge in the rear is located in front of the anus so as to prevent infection by bacteria.

10. The urine collector as claimed in claim 1, further comprising a layer of soft and absorbent wrapping material on the rim of the collecting cup which forms a web layer; the wrapping material also extends to the inward and downward area of the collecting cup, from the tapered edge of the collecting cup down to the lower opening of the collecting cup, to form a covering layer; when the collecting cup has been used, the user pushes the collecting cup along its tapered edge and squeezes the collecting cup, whereby a triangular piece is produced; the arc bottom of the triangular piece with a covering layer can seal the lower opening of the collecting cup which is installed above the slanted flexible neck, to prevent urine backflow from the lower opening of the collecting cup, and to further to absorb and wipe away any residual urine from leakage.
